(19) United States
(12) Patent Application Publication
(10) Pub. No.: US 2018/0117053 A1
(43) Pub. Date: May 3, 2018

(54) The Method of Use for Inhibitors of Epidermal Growth Factor Receptor Variants II, III and VI

(71) Applicant: Metastat, Inc., Boston, MA (US)

(72) Inventors: Elizabeth A. BUCK, Stony Brook, NY (US); Matthew O'CONNOR, Stony Brook, NY (US)

(73) Assignee: Metastat, Inc., Boston, MA (US)

(21) Appl. No.: 15/573,791

(22) PCT Filed: May 25, 2016
(86) PCT No.: PCT/US16/34090

§ 371 Date: Nov. 13, 2017

Related U.S. Application Data
(60) Provisional application No. 62/166,748, filed on May 27, 2015.

(51) Int. Cl. A61K 31/5377; A61K 31/435; A61K 31/517; A61P 35/00; G01N 33/50

(52) U.S. Cl. CPC A61K 31/5377; A61K 31/435; G01N 2333/71; A61P 35/00; G01N 33/5011; A61K 31/517

(57) ABSTRACT

A panel of 12 EGFR inhibitors were screened for inhibition of EGFR phosphorylation in U87MG tumor cells engineered to express EGFR-viii. Compounds elicited a range of activity against phosphoY1173-EGFR. While one group of inhibitors had relatively weak activity against EGFR-viii (WZ4002, WZ8040, WZ3146, CO-1686, and AZD9291) another group had relatively potent activity against EGFR-viii (pelitinib, canertinib, PD168393, neratinib, AST-1306, and dacomitininb). The data described herein provide new methods of use for pelitinib, canertinib, AST-1306, and PD168393 against cancer, such as GBM, that express EGFR-viii. Furthermore, neratinib also exhibited selectivity towards splice variants EGFR-vii and EGFR-vvi compared to wild-type EGFR.

### CROSS REFERENCE TO RELATED APPLICATIONS

This application claims priority to U.S. provisional application Ser. No. 62/166,748, filed May 27, 2015, the entire disclosure of which is incorporated by reference.

### TECHNICAL FIELD

The invention relates to treatments for cancer, and in particular for cancers expressing EGFR-viii, as well as EGFR splice variants EGFR-vii and EGFR-vvi.

### BACKGROUND OF THE INVENTION

The present application relates to methods of use for inhibitors of epidermal growth factor receptor variants ii, iii, and vi (EGFR-vii, EGFR-viii, EGFR-vvi). Identification of oncogenic mutations in non-small cell lung cancer (NSCLC) has transformed the treatment landscape for this disease. Patients whose tumors harbor constitutively activating mutations within the catalytic domain of EGFR, affecting exons 19, 20, and 21, receive initial benefit from EGFR kinase inhibitors including erlotinib and afatinib. See Chong and Janne, 2013.

Glioblastoma multiforme (GBM) represents another tumor type for which an oncogenic form of EGFR is expressed. However, in contrast to the activating mutations occurring at the catalytic domain of EGFR that are found in NSCLC, GBM tumors harbor variations affecting the ectodomain. See Brennan, Verhaak et al., 2013. The most common of these variations in GBM tumors is deletion of exons 2-7, encoding a region of the ectodomain which includes the first cysteine rich domain. This variant, termed EGFR variant iii (EGFR-viii) results from a coerced splicing event that occurs in conjunction with genomic amplification and rearrangement. See Sugawa, Ekstrand et al., 1990. EGFR-viii is constitutively dimerized, constitutively active, and is both transforming and tumorigenic for tumors including glioblastoma and breast See Nishikawa, Ji et al., 1994, Huang, Nagane et al., 1997, Tang, Gong et al., 2000. In GBM, expression of EGFR-viii is a negative prognostic indicator of long term overall survival. See Heimberger, Hlatky et al., 2005. GBM tumors also exhibit deletion of exons 14 and 15, known as EGFR variant ii (EGFR-vii), and deletion of exons 12 and 13, known as EGFR variant vi (EGFR-vvi).

Although significant progress has been made in treating NSCLC with reversible or covalent EGFR inhibitors, progress in treating GBM tumors with EGFR inhibitors has lagged. Clinical studies evaluating reversible EGFR inhibitors, including erlotinib, gefitinib, and lapatinib, or covalent EGFR inhibitors, including afatinib, have failed to demonstrate significant benefit for GBM. See Brandes, Franceschi et al., 2008, Vivanco, Robins et al., 2012, Reardon, Nabors et al., 2014. Selective inhibition of mutant forms of EGFR versus EGFR wild-type (WT) is predictive of clinical activity for EGFR inhibitors among NSCLC patients whose tumors express certain activating mutations in EGFR. See Barkovich, Hariono et al., 2012. For example, erlotinib has greater potency against the EGFR catalytic domain mutations EGFR-exon19 deletion and EGFR-L858R compared to EGFR-WT, which is consistent with clinical benefit for erlotinib within a NSCLC patient population whose tumors express these mutations. Selective inhibition of the EGFR drug resistance mutant EGFR-T790M is also predictive of sensitivity to covalent EGFR inhibitors within the population of NSCLC patients whose tumors express this mutation. Clinical studies have demonstrated greater response rates for CO-1686 and AZD9291 which are selective for EGFR-T790M, compared to afatinib and dacomitinib, which have no selectivity preference in favor of EGFR-T790M. See Steuer, Kimri, and Ramalingam et al., 2014.

Observations in NSCLC studies indicate that small molecule EGFR inhibitors that are both potent against EGFR-viii and selective for EGFR-viii versus EGFR-WT show greater activity compared to those small molecule inhibitors that do not have EGFR-viii selectivity. Previous studies have demonstrated that erlotinib has differential sensitivity against EGFR-viii compared to EGFR-WT or select EGFR mutants observed in NSCLC. Specifically these studies showed that erlotinib exhibited the following selectivity profile, with erlotinib being most potent against EGFR-exon19del and least potent EGFR-viii: EGFR-exon19del>EGFR-L858R>EGFR-WT>EGFR-viii. See Barkovich, Hariono et al., 2012. The lack of selective inhibition of EGFR-viii observed for erlotinib is in line with lack of significant clinical benefit among patients with GBM tumors, a subset of which express EGFR-viii.

### SUMMARY OF THE INVENTION

A method of inhibiting growth of tumor cells of a patient in need thereof is provided. The method comprises administering to the patient an effective amount of an EGFR inhibitor, wherein the tumor cells of the patient at least partially express a splice-activated variant of EGFR.

According to further embodiments: the splice-activated variant of EGFR is selected from the group consisting of EGFR variant ii (EGFR-vii), EGFR variant iii (EGFR-viii), and EGFR variant vi (EGFR-vvi); the EGFR inhibitor is selected from the group consisting of neratinib, pelitinib, canertinib, PD168393, and AST-1306; the EGFR inhibitor is at least five-fold selective for the splice-activated variant of EGFR versus EGFR wild type (EGFR-WT); the EGFR inhibitor is neratinib; the tumor cells are glioblastoma multiforme (GBM), squamous cell carcinoma of the head and neck (SCCHN), breast cancer, and lung cancer; the EGFR inhibitor has an EC50 of less than 50 nM against the splice-activated variant of EGFR; and the splice-activated variant of EGFR is inhibited by the EGFR inhibitor.

A method for treating cancer in a patient need thereof is provided. The method comprises obtaining a measurement from a sample of the patient's tumor cells, wherein the measurement indicates whether the tumor cells at least partially express a splice-activated variant of EGFR; and administering an effective amount of EGFR inhibitor to the patient if the patient's tumor cell expresses the splice-activated variant of EGFR.

According to further embodiments: the splice-activated variant of EGFR is selected from the group consisting of EGFR variant ii (EGFR-vii), EGFR variant iii (EGFR-viii), and EGFR variant vi (EGFR-vvi); the EGFR inhibitor is selected from the group consisting of neratinib, pelitinib, canertinib, PD168393, and AST-1306; the EGFR inhibitor is at least five-fold selective for the splice-activated variant of EGFR versus EGFR wild type (EGFR-WT); and the cancer is at least one of glioblastoma multiforme (GBM), squamous cell carcinoma of the head and neck (SCCHN), breast cancer, and lung cancer.

A method of screening inhibitors to determine whether the inhibitors inhibit growth of cancer expressing a splice-activated variant of EGFR is provided. The method comprises assessing an EGFR inhibitor's selectivity over a tumor cell expressing the splice-activated variant of EGFR versus a tumor cell expressing EGFR wild type (EGFR-WT); and determining that the EGFR inhibitor inhibits the growth of cancer expressing the splice-activated variant of EGFR when the EGFR inhibitor's selectivity over the tumor cell expressing the splice-activated variant of EGFR versus the tumor cell expressing EGFR-WT is above a predetermined threshold or determining that the EGFR inhibitor does not inhibit the growth of cancer expressing the splice-activated variant of EGFR when the EGFR inhibitor's selectivity over the tumor cell expressing the splice-activated variant of EGFR versus the tumor cell expressing EGFR-WT is below the predetermined threshold.

According to further embodiments: the predetermined threshold comprises at least a five-fold selectivity in the EGFR inhibitor's potency in the tumor cell expressing the splice-activated variant of EGFR over the tumor cell expressing EGFR-WT; the predetermined threshold comprises at least a ten-fold selectivity in the EGFR inhibitor's potency in the tumor cell expressing the splice-activated variant of EGFR over the tumor cell expressing EGFR-WT; the cancer expressing the splice-activated variant of EGFR is selected from the group consisting of glioblastoma multiforme (GBM), squamous cell carcinoma of the head and neck (SCCHN), breast cancer, and lung cancer; and the splice-activated variant of EGFR is selected from the group consisting of EGFR-vii, EGFR-viii, EGFR-vvi and EGFR-T790M.

A method of treating a disease or disorder of a patient in need thereof is provided. The method comprises administering to the patient an effective amount of an EGFR inhibitor, wherein the disease or disorder of the patient is associated with expression of a splice-activated variant of EGFR.

According to further embodiments: the EGFR inhibitor is selected from the group consisting of neratinib, pelitinib, canertinib, PD168393, and AST-1306; the EGFR inhibitor is at least five-fold selective for the splice-activated variant of EGFR versus EGFR wild type (EGFR-WT); and the splice-activated variant of EGFR is selected from the group consisting of EGFR-vii, EGFR-viii, EGFR-vvi and EGFR-T790M.

### BRIEF DESCRIPTION OF THE DRAWINGS

The above and other aspects, features and advantages of certain embodiments will be more apparent from the following detailed description taken in conjunction with the accompanying drawings, in which:

FIG. 1 illustrates a graph of EC50 data for EGFR inhibitors pelitinib, afatinib, canertinib, dacomitinib, PD168393, neratinib, AST-1306, AZD9291, WZ3146, WZ4002, WZ8040, and CO-1686 against phosphoY1173-EGFRviii expressed in U87MG tumor cells;

FIG. 2 illustrates a graph for determining whether EGFR tyrosine kinase inhibitors inhibit cancer growth in tumors expressing mutational or splicing variants of EGFR, including EGFR-T790M and EGFR-viii, based on selectivity versus EGFR-WT;

FIG. 3A illustrates a table of potency and selectivity data for reversible (lapatinib and TAK-285) and covalent (afatinib and neratinib) EGFR inhibitors against pY1173-EGFR in U87MG cells engineered to express either EGFR-WT or EGFR-viii;

FIG. 3B illustrates a graph demonstrating the effect of varying concentrations of neratinib on phosphoY1173-EGFR-viii or phosphoY1173-EGFR-WT in U87MG tumor cells expressing either EGFR-viii or EGFR-WT, respectively;

FIG. 3C illustrates a graph of the selectivity of TAK-285 and neratinib for EGFR-viii within the selectivity model for predicting clinical benefit illustrated by FIG. 2; and

FIG. 4 illustrates a table of potency data for neratinib against EGFR-vii, EGFR-viii, and EGFR-vvi and relative selectivity of neratinib to each of EGFR-vii, EGFR-viii, and EGFR-vvi compared to EGFR-WT.

### DETAILED DESCRIPTION

The following detailed description of certain embodiments will be made in reference to the accompanying drawings. In the detailed description, explanation about related functions or constructions known in the art are omitted for the sake of clearness in understanding the concept of the invention, to avoid obscuring the invention with unnecessary detail.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

The terms “patient” and “subject” refer to a human in need of treatment with an EGFR kinase inhibitor for any purpose, and to a human in need of such a treatment to treat cancer, or a precancerous condition or lesion. However, the terms “patient” and “subject” can also refer to non-human animals, preferably mammals such as dogs, cats, horses, cows, pigs, sheep and non-human primates, among others, that are in need of treatment with an EGFR kinase inhibitor. In a preferred embodiment, the patient is a human in need of treatment for cancer, a precancerous condition or lesion, or other forms of abnormal cell growth. The cancer is any cancer treatable, either partially or completely, by administration of an EGFR kinase inhibitor. The cancer may be, for example, lung cancer, non-small cell lung cancer (NSCLC), bronchioloalviolar cell lung cancer, bone cancer, pancreatic cancer, skin cancer, cancer of the head or neck, cutaneous or introcular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyopsis colon cancer syndrome (HNPCC), Barrett's esophagus, bladder dysplasia, and precancerous cervical conditions.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

A panel of 12 EGFR inhibitors was screened for inhibition of EGFR phosphorylation in U87MG tumor cells engineered to express EGFR-viii. Compounds elicited a range of activity against phosphoY1173-EGFR. The data described herein supports new methods of use for neratinib, pelitinib, canertinib, AST-1306, and PD168393 against cancer, such as GBM that express EGFR-viii. Several recent studies have demonstrated that EGFR inhibitors including dacomitinib, afatinib, and neratinib are potent inhibitors of EGFR-viii. See Ji, Zhao et al., 2006, Vivanco, Robins et al., 2012. However, prior studies have not included broad scale profiling of other EGFR inhibitors against EGFR variant ii (EGFR-vii), EGFR variant iii (EGFR-viii) and EGFR variant vi (EGFR-vvi).

The pharmaceutically active compounds described herein active as EGFR-vii, EGFR -viii and EGFR-vvi kinase inhibitors (collectively referred to herein as “mutant EGFR inhibitors”, and thus, they exhibit therapeutic utility in treating cancer. The mutant EGFR inhibitors described herein are useful for the treatment of a disease or disorder selected from cancer, such as glioblastoma multiforme (GBM), including giant cell glioblastoma and gliosarcoma, squamous cell carcinoma of the head and neck (SCCHN), breast cancer, and lung cancer. See Tang, Gong et al., 2000, Okamoto, Kenyon et al., 2003, Rae, Scheys et al., 2004, Wheeler, Suzuki et al., 2010. The pharmaceutically active compounds described herein are useful as mutant EGFR inhibitors in mammals, particularly humans, in need thereof

Accordingly, a method of treating tumor cells of a patient in need thereof includes administering to the patient an effective amount of an EGFR inhibitor, wherein the tumor cells of the patient express a variant of EGFR including at least one of EGFR-vii, EGFR-viii and EGFR-vvi.

A method is provided for treating tumor cells of a patient in need thereof. The method includes contacting tumor cells of the patient with an effective amount of an EGFR inhibitor, wherein the tumor cells of the patient express at least one of EGFR-vii, EGFR-viii and EGFR-vvi.

A method is provided for treating tumor cells of a patient in need thereof. The method includes modulating activity of at least one of EGFR-vii, EGFR-viii and EGFR-vvi in the patient by administering an effective amount of an EGFR inhibitor.

A method is provided for treating a disease, disorder, symptom, or condition associated with expression of at least one of EGFR-vii, EGFR-viii and EGFR-vvi in a patient in need. The method includes administering to the patient an effective amount of a pharmaceutical composition including an EGFR inhibitor or a pharmaceutically acceptable salt thereof.

A method is provided for screening inhibitors to determine whether the inhibitors inhibit growth of cancer expressing a variant of EGFR including at least one of EGFR-vii, EGFR-viii, and EGFR-vvi. The method includes contacting a sample of a tumor cell expressing the variant of EGFR from a subject with an EGFR inhibitor; measuring potency of the EGFR inhibitor against the variant of EGFR; contacting a sample of a tumor cell expressing EGFR wild type (EGFR-WT) from a subject with the EGFR inhibitor; measuring potency of the EGFR inhibitor against EGFR-WT; assessing the EGFR inhibitor's selectivity over the tumor cell expressing the variant of EGFR and the tumor cell expressing EGFR-WT; and determining that the EGFR inhibitor inhibits the growth of cancer expressing the variant of EGFR when the EGFR inhibitor's selectivity over the tumor cell expressing the variant of EGFR and the tumor cell expressing EGFR-WT is above a predetermined threshold or determining that the EGFR inhibitor does not inhibit the growth of cancer expressing the variant of EGFR when the EGFR inhibitor's selectivity over the tumor cell expressing the variant of EGFR and the tumor cell expressing EGFR-WT is below the predetermined threshold.

The mutant EGFR inhibitors described herein may be administered orally or parenterally. The mutant EGFR inhibitors and other additional agents can he administered in single or multiple doses. The mutant EGFR inhibitors can be administered with pharmaceutically acceptable salts and with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored. The mutant EGFR inhibitors can be combined together with various pharmaceutically acceptable inert carriers in the form of sprays, creams, salves, suppositories, jellies, gels, pastes, lotions, ointments, and the like. Administration -of such dosage forms can be carried out in single or multiple doses. Carriers include solid diluents or fillers, sterile aqueous media, and various non-toxic organic solvents, etc. The effectiveness of treatment in the preceding methods can, for example, be determined by measuring the decrease in size of tumors present in the patients, or by assaying a molecular determinant of the degree of proliferation of the tumor cells.

Dosage levels for the mutant EGFR inhibitors are as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The mutant EGFR inhibitors are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

Pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the mutant EGFR inhibitors are administered in a pharmaceutical dosage unit and will be an efficacious, nontoxic quantity selected from the range of 0.001-100 mg/kg of active compound. When treating a human patient in need of an EGFR inhibitor, the selected dose is administered from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

### EXAMPLES

### Generation of U87MG Tumor Cells Expressing EGFR-WT, EGFR-vii, EGFR-viii, or EGFR-vvi:

U87MG tumor cells (ATCC) were cultured in media recommended by ATCC and were engineered to express either EGFR-WT, EGFR-vii, EGFR-viii or EGFR-vvi through lentiviral infection followed by puromycin selection. Cells stably expressing EGFR-WT, EGFR-vii, EGFR-viii or EGFR-vvi were maintained in the presence of 0.5 μg/ml puromycin.

### Preparation of Compounds:

All EGFR inhibitors were purchased from Selleck Chemicals, Houston TX, at a concentration of 10 mM in DMSO. Experimental procedures for synthesis of EGFR inhibitors are known in the art. Serial dilution of compounds was prepared using DMSO at 100X the indicated final concentration and then diluted in cell media to the indicated final concentration.

### Determination of PhosphoY1173-EGFR:

Measurements by in cell ELISA were determined using the EGFR Colorimetric In-Cell ELISA Kit (62205, Thermo-Scientific, Rockford Ill.) according to manufacturer's procedure. Measurements by western blot were determined by preparing cell extracts using RIPA extraction buffer (R0278, Sigma, St. Louis Mo.) supplemented with protease and phosphatase inhibitors (P8340, Sigma, St. Louis Mo.), followed by electrophoretic transfer of SDS-PAGE separated proteins to nitrocellulose and detection using anti-phosphoY1173-EGFR antibody (#53A5, Cell Signaling Technologies, Danvers, Mass.) and chemiluminescent detection (Thermo-Scientific, Rockford Ill.).

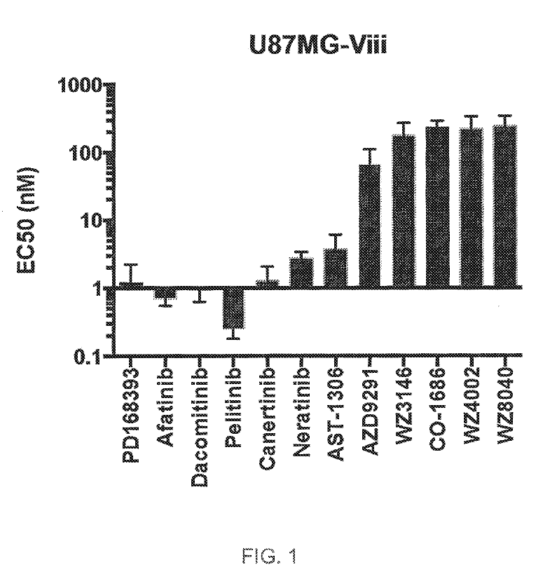

FIG. 1 illustrates a graph of EC50 data for covalent EGFR inhibitors pelitinib, afatinib, canertinib, dacomitinib, PD168393, neratinib, AST-1306, AZD9291, WZ3146, WZ4002, WZ8040, and CO-1686 against phosphoY1173-EGFRviii expressed in U87MG tumor cells. Data are expressed as EC50 and were determined using an in-cell ELISA kit (Pierce). Experimental procedures for synthesis of these EGFR inhibitors are known in the art.

As illustrated by FIG. 1, several inhibitors with previously un-described EGFR-viii activity are potent inhibitors of EGFR-viii. While WZ4002, WZ8040, WZ3146, CO-1686, and AZD9291 had weak activity against EGFR-viii, pelitinib, canertinib, PD168393, neratinib, AST-1306, and dacomitinib were potent against EGFR-viii. That is, pelitinib, canertinib, PD168393, neratinib, AST-1306, and dacomitinib were found to have EC50 values of less than 50 nM against EGFR-viii, and thus, were at least ten-fold more potent against EGFR-viii than WZ4002, WZ8040, WZ3146, CO-1686, and AZD9291.

Prior studies have not addressed the selectivity of EGFR inhibitors for EGFR-viii versus EGFR-WT. Data provided herein indicates that an EGFR inhibitor's selectivity toward an EGFR variant over EGFR-WT above a predetermined threshold determines whether the EGFR inhibitor inhibits growth of cancer expressing the EGFR variant.

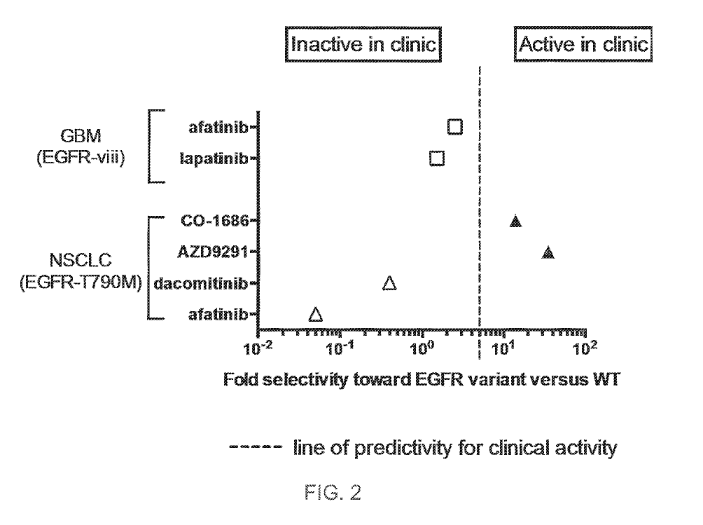

FIG. 2 illustrates a graph for determining whether EGFR tyrosine kinase inhibitors inhibit cancer growth in tumors expressing mutational or splicing variants of EGFR, including EGFR-T790M and EGFR-viii, based on selectivity versus EGFR-WT. Molecules with insufficient selectivity, i.e., less than five-fold, for an EGFR variant versus EGFR-WT are inactive at inhibiting cancer growth. Molecules with sufficient selectivity, i.e., greater than five-fold, for an EGFR variant versus EGFR-WT inhibit cancer growth. Accordingly, a threshold of greater than five-fold selectivity for a variant of EGFR versus EGFR-WT determines whether an EGFR inhibitor inhibits cancer growth in tumors expressing the EGFR variant.

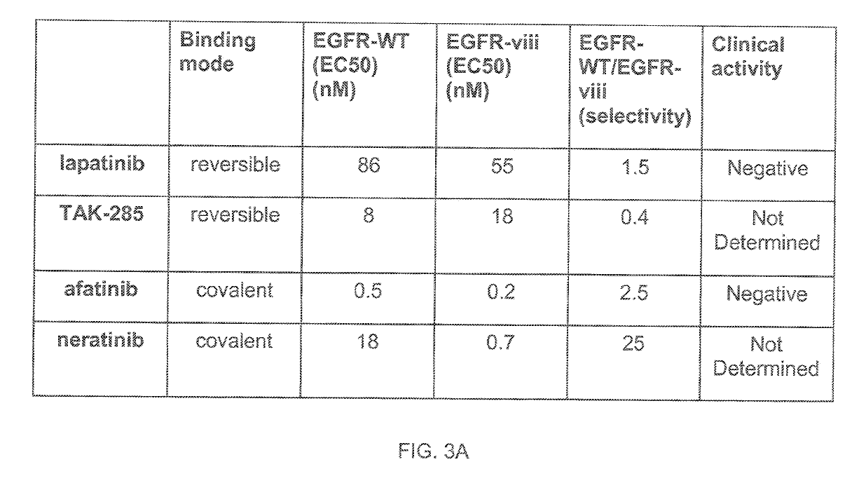

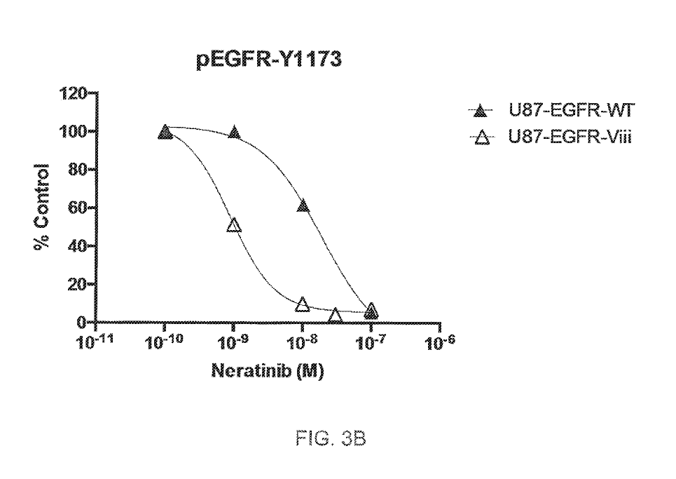

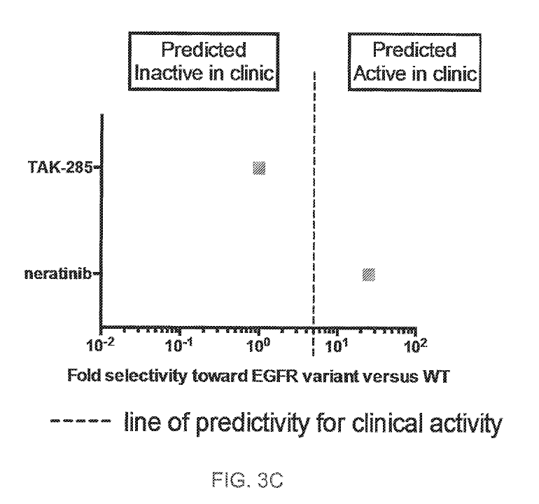

FIG. 3A illustrates a table of potency and selectivity data for reversible (lapatinib and TAK-285) and covalent (afatinib and neratinib) EGFR inhibitors against pY1173-EGFR in U87MG cells engineered to express either EGFR-WT or EGFR-viii. Lapatinib, TAK-285, and afatinib demonstrate selectivity of less than five-fold. Lapatinib and afatinib have demonstrated insignificant cancer growth inhibition in clinical studies. See Vivanco, Robins et al., 2012, Reardon, Nabors et al., 2014. Based on having less than five-fold selectivity for EGFR-viii, TAK-285 will demonstrate insignificant cancer growth inhibition in tumors expressing EGFR-viii. Neratinib exhibits greater than five-fold selectivity, e.g., 25-fold selectivity, toward EGFR-viii versus EGFR-WT and will therefore demonstrate significant cancer growth inhibition when administered to patient with tumors expressing EGFR-viii.

FIG. 3B illustrates a graph demonstrating the effect of varying concentrations of neratinib on phosphoY1173-EGFR-viii or phosphoY1173-EGFR-WT in U87MG tumor cells expressing either EGFR-viii or EGFR-WT, respectively. Data are expressed as EC50 and were determined by western blotting using an anti-pY1173-EGFR antibody (Cell Signaling Technologies).

FIG. 3C illustrates a graph of TAK-285 and neratinib overlaid on the graph of FIG. 2. Thus, it is demonstrated that neratinib is greater than five-fold selective for EGFR-viii expressing tumor cells versus tumor cells expressing EGFR-WT. Accordingly, neratinib inhibits cancer growth in tumors expressing EGFR-viii.

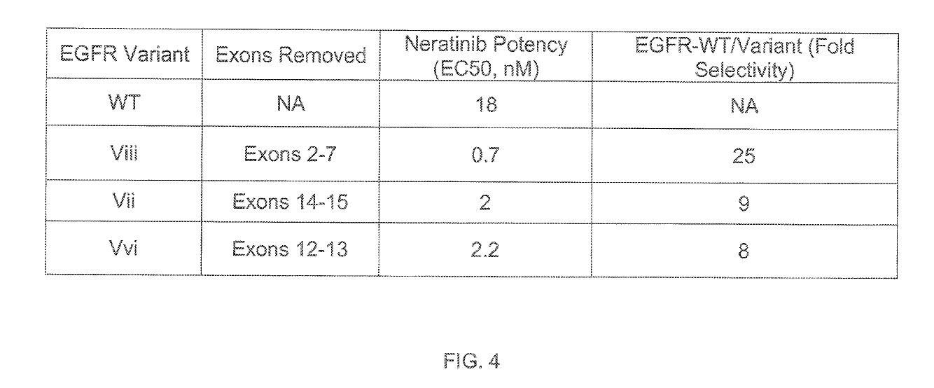

FIG. 4 illustrates a table of potency data for neratinib against EGFR-vii, EGFR-viii, and EGFR-vvi and relative selectivity of neratinib to each of EGFR-vii, EGFR-viii, and EGFR-vvi compared to EGFR-WT The data provided in FIG. 4 indicate that neratinib exhibits greater than five-fold selectivity toward EGFR-vii and EGFR-vvi versus EGFR-WT and that neratinib is selective for all splice variants of EGFR, e.g., EGFR-vii, EGFR-viii, and EGFR-vvi, compared with EGFR-WT. Thus, neratinib will demonstrate significant cancer growth inhibition when administered to patients with tumors expressing at least one of EGFR-vii, EGFR-viii and EGFR-vvi.

While embodiments of the invention have been shown and described with reference to certain embodiments thereof, it will be understood by those skilled in the art that various changes in form and details may be made therein without departing from the spirit and scope of the invention as defined by the appended claims and equivalents thereof.

### REFERENCES

Barkovich, K. J., S. Hariono, A. L. Garske, J. Zhang, J. A. Blair, Q. W. Fan, K. M. Shokat T. Nicolaides and W. A. Weiss (2012). “Kinetics of inhibitor cycling underlie therapeutic disparities between EGFR-driven lung and brain cancers.” Cancer Discov 2(5): 450-457.

Brandes, A. A., E. Franceschi, A. Tosoni, M. E. Hegi and R. Stupp (2008). “Epidermal growth factor receptor inhibitors in neuro-oncology: hopes and disappointments.” Clin Cancer Res 14(4): 957-960.

Brennan, C. W., R. G. Verhaak, A. McKenna, B. Campos, H. Noushmehr, S. R. Salama, S. Zheng, D. Chakravarty, J. Z. Sanborn, S. H. Berman, R. Beroukhim, B. Bernard, C. J. Wu, G. Genovese, I. Shmulevich, J. Bamholtz-Sloan, L. Zou, R. Vegesna, S. A. Shukla, G. Ciriello, W. K. Yung, W. Zhang, C. Sougnez, T. Mikkelsen, K. Aldape, D. D. Bigner, E. G. Van Meir, M . Prados, A. Sloan, K. L. Black, J. Eschbacher, G. Finocchiaro, W. Friedman, D. W. Andrews, A. Guha, M. Iacocca, B. P. O'Neill, G. Foltz, J. Myers, D. J. Weisenberger, R. Penny, R. Kucherlapati, C. M. Perou, D. N. Hayes, R. Gibbs, M. Marra, G. B. Mills, E. Lander, P. Spellman, R. Wilson, C. Sander, J. Weinstein, M. Meyerson, S. Gabriel, P. W. Laird, D. Haussler, G. Getz, L. Chin and T. R. Network (2013). “The somatic genomic landscape of glioblastoma.” Cell 155(2): 462-477.

Chong, C. R. and P. A. Janne (2013). “The quest to overcome resistance to EGFR-targeted therapies in cancer.” Nat Med 19(11): 1389-1400.

Heimberger, A . B., R. Hlatky, D. Suki, D. Yang, J. Weinberg, M. Gilbert, R. Sawaya and K. Aldape (2005). “Prognostic effect of epidermal growth factor receptor and EGFR vIII in glioblastoma multiforme patients.” Clin Cancer Res 11(4): 1462-1466.

Huang, H. S., M. Nagane, C. K. Klingbeil, H. Lin, R. Nishikawa, X. D. Ji, C. M. Huang, G. N. Gill, H. S. Wiley and W. K. Cavenee (1997). “The enhanced tumorigenic activity of a mutant epidermal growth factor receptor common in human cancers is mediated by threshold levels of constitutive tyrosine phosphorylation and unattenuated signaling.” J Biol Chem 272(5): 2927-2935.

Ji, H., X. Zhao, Y. Yuza, T. Shimarnura, D. Li, A. Protopopov, B. L. Jung, K. McNamara, H. Xia, K. A. Glatt, R. K. Thomas, H. Sasaki, J. W. Homer, M. Eck, A. Mitchell, Y. Sun, R. Al-Hashem, R. T. Bronson, S. K. Rabindran, C M. Discafani, E. Maher, G. I Shapiro, M. Meyerson and K. K. Wong (2006). “Epidermal growth factor receptor variant III mutations in lung tumorigenesis and sensitivity to tyrosine kinase inhibitors.” Proc Natl Acad Sci U S A 103(20): 7817-7822.

Nishikawa, R., X. D. Ji, R. C. Harmon, C. S. Lazar, G. N. Gill, W K. Cavenee and H. J. Huang (1994). “A mutant epidermal growth factor receptor common in human glioma confers enhanced tumorigenicity.” Proc Natl Acad Sci U S A 91(16): 7727-7731.

Okamoto, I., L C. Kenyon, D. R. Emlet, T. Mori, J . Sasaki, S. Hirosako, Y. Ichikawa, H. Kishi, A. K. Godwin, M. Yoshioka, M. Suga, M. Matsumoto and A. J. Wong (2003). “Expression of constitutively activated EGFR vIII in non-small cell lung cancer.” Cancer Sci 94(1): 50-56.

Rae, J. M., J. O. Scheys, K. M. Clark, R. B. Chadwick, M . C. Kiefer and M. E. Lippman (2004). “EGFR and EGFR vIII expression in primary breast cancer and cell lines.” Breast Cancer Res Treat 87(1): 87-95.

Reardon, D. A., L. B. Nabors, W. P. Mason, J. R. Perry, W. Shapiro, P. Kavan, D. Mathieu, S. Phuphanich, A. Cseh, Y. Fu, J. Cong, S. Wind, D. D. Eisenstat, B. I. T. G. on behalf of the and C. the Canadian Brain Tumour (2014). “Phase I/randomized phase II study of afatinib, an irreversible ErbB family blocker, with or without protracted temozolomide in adults withrecurrent glioblastoma.” Neuro Oncol.

Steuer, C. E., F. R. Kimri and S. S. Ramalingam (2014). “The next generation of epidermal growth factor receptor tyrosine kinase inhibitors in the treatment of lung cancer.” Cancer.

Sugawa, N., A. J. Ekstrand, C. D. James and V. P. Collins (1990). “Identical splicing of aberrant epidermal growth factor receptor transcripts from amplified rearranged genes in human glioblastomas.” Proc Natl Acad Sci U S A 87(21): 8602-8606.

Tang, C. K., X. Q. Gong, D. K. Moscatello, A. J. Wong and M. E. Lippman (2000). “Epidermal growth factor receptor vIII enhances tumorigenicity in human breast cancer.” Cancer Res 60(1 1): 3081-3087.

Vivanco, I., H. I. Robins, D. Rohle, C. Campos, C. Grommes, P. L. Nghiempbu, S. Kubek, B. Oldrini, M. G. Chheda, N. Yannuzzi, H. Tao, S. Zhu, A. lwanami, D. Kuga, J. Dang, A. Pedraza, C. W. Brennan, A. Heguy, L. M. Liau, F. Lieberman, W. K. Yung, M. R. Gilbert, D. A. Reardon, J. Drappatz, P. Y. Wen, K. R. Lamborn, S. M. Chang, M. D. Prados, H. A. Fine, S. Horvath, N. Wu, A . B. Lassrnan, L. M. DeAngelis, W. H . Yong, J G. Kuhn, P. S. Mischel, M. P. Mehta, T. F. Cloughesy and I. K . Mellinghoff (2012). “Differential sensitivity of glioma-versus lung cancer-specific EGFR mutations to EGFR kinase inhibitors.” Cancer Discov 2(5): 458-471.

Wheeler, S. E., S. Suzuki, S. M . Thomas, M . Sen, R. J. Leeman-Neill, S. I. Chiosea, C. T. Kuan, D. D. Bigner, W. E. Gooding, S. Y. Lai and J. R. Grandis (2010). “Epidermal growth factor receptor variant HI mediates head and neck cancer cell invasion via STAT3 activation.” Oncogene 29(37): 5135-5145.

What is claimed is:

1. A method of inhibiting growth of tumor cells of a patient in need thereof: the method comprising administering to the patient an effective amount of an EGFR inhibitor, wherein the tumor cells of the patient at least partially express a splice-activated variant of EGFR.

2. The method according to claim 1, wherein the splice-activated variant of EGFR is selected from the group consisting of EGFR variant ii (EGFR-vii), EGFR variant iii (EGFR-viii), and EGFR variant vi (EGFR-vvi).

3. The method according to claim 1, wherein the EGFR inhibitor is selected from the group consisting of neratinib, pelitinib, canertinib, PD168393, and AST-1306.

4. The method according to claim 1, wherein the EGFR inhibitor is at least five-fold selective for the splice-activated variant of EGFR versus EGFR wild type (EGFR-WT).

5. The method according to claim 4, wherein the EGFR inhibitor is neratinib.

6. The method according to claim 1, wherein the tumor cells are glioblastoma multiforme (GBM), squamous cell carcinoma of the head and neck (SCCHN), breast cancer, and lung cancer.

7. The method according to claim 1, wherein the EGFR inhibitor has an EC50 of less than 50 nM against the splice-activated variant of EGFR.

8. The method according to claim 1, wherein the splice-activated variant of EGFR is inhibited by the EGFR inhibitor.

9. A method for treating cancer in a patient need thereof, comprising:
obtaining a measurement from a sample of the patient's tumor cells, wherein the measurement indicates whether the tumor cells at least partially express a splice-activated variant of EGFR: and
administering an effective amount of EGFR inhibitor to the patient if the patient's tumor cell expresses the splice-activated variant of EGFR.

10. The method according to claim 9, wherein the splice-activated variant of EGFR is selected from the group consisting of EGFR variant ii (EGFR-vii), EGFR variant iii (EGFR-viii), and EGFR variant vi (EGFR-vvi).

11. The method according to claim 9, wherein the EGFR inhibitor is selected from the group consisting of neratinib, pelitinib, canertinib, PD168393, and AST-1306.

12. The method according to claim 9, wherein the EGFR inhibitor is at least five-fold selective for the splice-activated variant of EGFR versus EGFR wild type (EGFR-WT).

13. The method according to claim 9, wherein the cancer is at least one of glioblastoma multiforme (GBM), squamous cell carcinoma of the head and neck (SCCHN), breast cancer, and lung cancer.

14. A method of screening inhibitors to determine whether the inhibitors inhibit growth of cancer expressing a splice-activated variant of EGFR, the method comprising:
assessing an EGFR inhibitor's selectivity over a tumor cell expressing the splice-activated variant of EGFR versus a tumor cell expressing EGFR wild type (EGFR-WT); and
determining that the EGFR inhibitor inhibits the growth of cancer expressing the splice-activated variant of EGFR when the EGFR inhibitor's selectivity over the tumor cell expressing the splice-activated variant of EGFR versus the tumor cell expressing EGFR-WT is above a predetermined threshold or determining that the EGFR inhibitor does not inhibit the growth of cancer expressing the splice-activated variant of EGFR when the EGFR inhibitor's selectivity over the tumor cell expressing the splice-activated variant of EGFR versus the tumor cell expressing EGFR-WT is below the predetermined threshold.

15. The method according to claim 14, wherein the predetermined threshold comprises at least a five-fold selectivity in the EGFR inhibitor's potency in the tumor cell expressing the splice-activated variant of EGFR over the tumor cell expressing EGFR-WT.

16. The method according to claim 14, wherein the predetermined threshold comprises at least a ten-fold selectivity in the EGFR inhibitor's potency in the tumor cell expressing the splice-activated variant of EGFR over the tumor cell expressing EGFR-WT.

17. The method according to claim 14, wherein the cancer expressing the splice-activated variant of EGFR is selected from the group consisting of glioblastoma multiforme (GBM), squamous cell carcinoma of the head and neck (SCCHN), breast cancer, and lung cancer.

18. The method according to claim 14, wherein the splice-activated variant of EGFR is selected from the group consisting of EGFR-vii, EGFR-viii, EGFR-vvi and EGFR-T790M.

19. A method of treating a disease or disorder of a patient in need thereof, the method comprising administering to the patient an effective amount of an EGFR inhibitor, wherein the disease or disorder of the patient is associated with expression of a splice-activated variant of EGFR.

20. The method according to claim 19, wherein the EGFR inhibitor is selected from the group consisting of neratinib, pelitinib, canertinib, PD168393, and AST-1306.

21. The method according to claim 19, wherein the EGFR inhibitor is at least five-fold selective for the splice-activated variant of EGFR versus EGFR wild type (EGFR-WT).

22. The method according to claim 19, wherein the splice-activated variant of EGFR is selected from the group consisting of EGFR-vii, EGFR-viii, EGFR-vvi and EGFR-T790M.
